(19) United States
(12) Patent Application Publication
(10) Pub. No.: US 2014/0343646 A1
(43) Pub. Date: Nov. 20, 2014

(54) DUAL SIDE LOAD ANCHOR FOR ELECTRODE LEAD, SYSTEMS CONTAINING THE ANCHOR, AND METHODS OF MAKING AND USING

(71) Applicant: BOSTON SCIENTIFIC NEUROMODULATION CORPORATION, Valencia, CA (US)

(72) Inventor: Jacob B. Leven, Huntington Beach, CA (US)

(73) Assignee: BOSTON SCIENTIFIC NEUROMODULATION CORPORATION, Valencia, CA (US)

(21) Appl. No.: 14/263,971

(22) Filed: Apr. 28, 2014

Related U.S. Application Data
(60) Provisional application No. 61/823,729, filed on May 15, 2013.

(51) Int. Cl. A61N 1/05

(52) U.S. Cl. CPC A61N 1/05
USPC 607/116

(57) ABSTRACT

An implantable lead anchor includes a first anchor shell and a second anchor shell. The first and second anchor shells define two parallel lead channels. The lead anchor also includes an actuating mechanism to operably adjust the first and second anchor shells between a load position and a lock position. In the lock position, the first and second anchor shells are closer together than in the load position and any lead disposed within one of She two parallel lead channels is locked in place. In the load position, the first and second anchor shells are sufficiently spaced apart so that a portion of a lead can be side-loaded between the first and second anchor shells. The lead anchor also includes at least one guide element that extends between the first and second anchor shells to maintain an orientation of the first and second anchor shells relative to each other.

### CROSS-REFERENCE TO RELATED APPLICATIONS

This application claims the benefit under 35 U.S.C. §119(e) of U.S. Provisional Patent Application Ser. No. 61/823,729, filed May 15, 2013, which is incorporated herein by reference.

### FIELD

The present invention is directed to the area of implantable electrical stimulation systems and methods of making and using the systems. The present invention is also directed implantable electrical stimulation leads having lead anchors as well as methods of making and using the lead anchors, the leads and electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the nerves, muscles, or other tissue to be stimulated. The pulse generator in the control module generates electrical pulses that are delivered by the electrodes to body tissue. A lead anchor is often used to anchor the lead, thereby providing sufficient grip to the lead in order to keep the lead in a correct position with respect to the patient.

### BRIEF SUMMARY

One embodiment is an implantable lead anchor that includes a first anchor shell and a second anchor shell disposed opposite the first anchor shell. The first and second anchor shells define two parallel lead channels between the first and second anchor shells. The lead anchor also includes an actuating mechanism to operably adjust the first and second anchor shells between a load position and a lock position, in the lock position, the first and second anchor shells are closer together than in the load position and any lead disposed within one of the two parallel lead channels is locked in place. In the load position, the first and second anchor shells are sufficiently spaced apart so that a portion of a lead can be side-loaded between the first and second anchor shells and into one of the two parallel lead channels. The lead anchor also includes at least one guide element that extends between the first and second anchor shells and is configured and arranged to maintain an orientation of the first and second anchor shells relative to each other in the lock position, in the load position, and during adjustment between the lock and load positions.

Another embodiment is a kit including the implantable lead anchor described above and at least one electrical stimulation lead. The implantable lead anchor is configured and arranged to receive a portion of one of the at least one electrical stimulation lead in one of the two parallel lead channels.

A further embodiment is a method of implanting an electrical stimulation lead. The method includes side-loading a portion of a first electrical stimulation lead into one of the two lead channels of the implantable lead anchor described above, with the first and second anchor shells of the implantable lead anchor in the load position; and operating the actuating mechanism to adjust the first and second anchor shells to the lock position to lock the first electrical stimulation lead in the implantable lead anchor.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a belter understanding of the present invention, reference will be made to the following Detailed Description, which is to be read in association with the accompanying drawings, wherein:

FIG. 1 is a schematic side view of one embodiment of an electrical stimulation system, that includes a paddle lead electrically coupled to a control module, according to the invention;

FIG. 2 is a schematic side view of one embodiment of an electrical stimulation system that includes a percutaneous lead electrically coupled to a control module, according to the invention;

FIG. 3A is a schematic side view of one embodiment of the control module of FIG. 1 configured and arranged to electrically couple to an elongated device, according to the invention;

FIG. 3B is a schematic side view of one embodiment of a lead extension configured and arranged to electrically couple the elongated device of FIG. 2 to the control module of FIG. 1, according to the invention;

FIG. 4A is a schematic perspective view of one embodiment of a dual side-loading lead anchor in a load position; according to the invention;

FIG. 4B is a schematic perspective view of the dual side-loading lead anchor of FIG. 4A in a lock position; according to the invention;

FIG. 4C is a schematic cross-sectional view of the dual side-loading lead anchor of FIG. 4A in the load position: according to die invention;

FIG. 4D is a schematic cross-sectional view of the dual side-loading lead anchor of FIG. 4A in the lock position; according to the invention;

FIG. 5A is a schematic perspective view of another embodiment of a dual side-loading lead anchor in a load position; according to the invention;

FIG. 5B is a schematic perspective view of the dual side-loading lead anchor of FIG. 5A in a lock position; according to the invention;

FIG. 5C is a schematic cross-sectional view of the dual side-loading lead anchor of FIG. 5A in the load position; according to the invention;

FIG. 5D is a schematic cross-sectional view of the dual side-loading lead anchor of FIG. 5A in the lock position; according to the invention; and

FIG. 6 is a schematic overview of one embodiment of components of a stimulation system, including an electronic subassembly disposed within a control module, according to the invention.

### DETAILED DESCRIPTION

The present invention is directed to the area of implantable electrical stimulation systems and methods of making and using the systems. The present invention is also directed implantable electrical stimulation leads having lead anchors as well as methods of making and using the lead anchors, the leads and electrical stimulation systems.

Suitable implantable electrical stimulation systems include, but are not limited to, a least other lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example. U.S. Pat. Nos. 6,181,969; 6,516.227; 6,609,029; 6,609,032; 6,741,892; 7,949,395; 7,244,150: 7,672,734; 7,761,165; 7,974,706; 8,175,710; 8,224,450; and 8,364,278; and U.S. Patent Application Publication No. 2007/0150036, all of which are incorporated by reference.

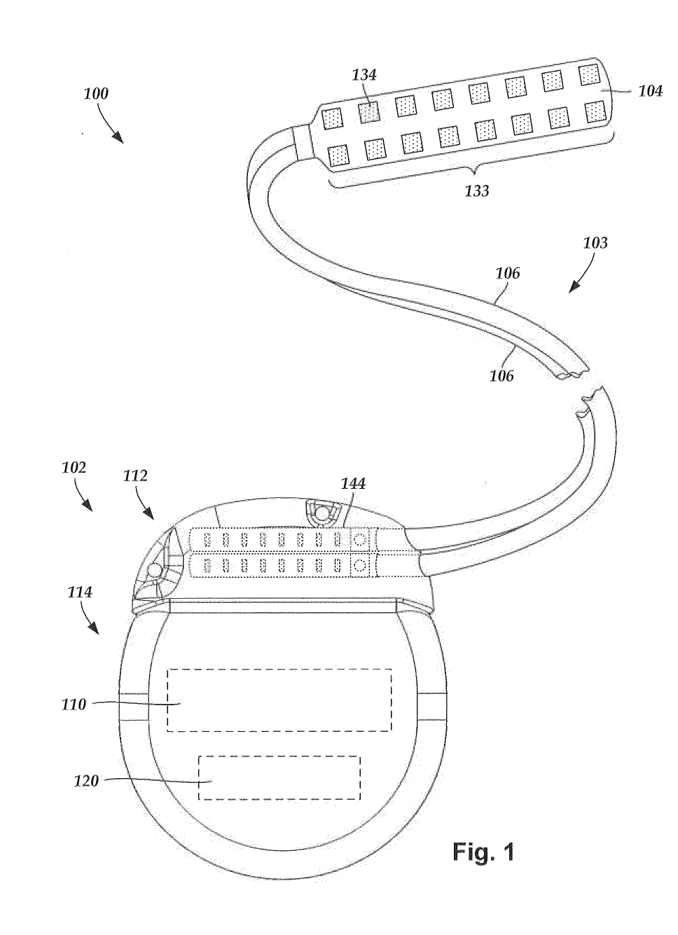

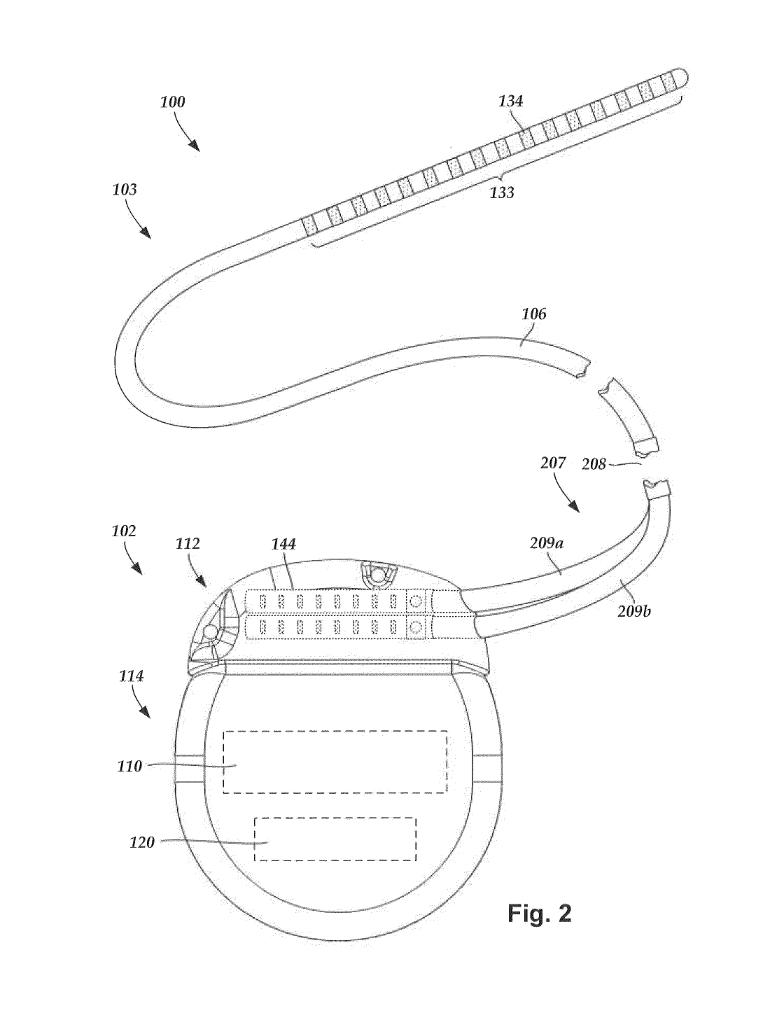

FIG. 1 illustrates schematically one embodiment of an electrical stimulation system 100. The electrical stimulation system includes a control module (e.g., a stimulator or pulse generator) 102 and a lead 103 coupleable to the control module 102. The lead 103 includes a paddle body 104 and one or more lead bodies 106. In FIG. 1, the lead 103 is shown leaving two lead bodies 106. It will be understood that the lead 103 can include any suitable number of lead bodies including, for example, one, two, three, four, five, six, seven, eight or more lead bodies 106. An array of electrodes 133, such as electrode 134, is disposed on the paddle body 104, and an array of terminals (e.g., 210 in FIG. 2A-2B) is disposed along each of the one or more lead bodies 106.

It will be understood that the electrical stimulation system can include more, fewer, or different components and can have a variety of different configurations including those configurations disclosed in the electrical stimulation system references cited herein. For example, instead of a paddle body, the electrodes can be disposed in an array at or near the distal end of a lead body forming a percutaneous lead.

FIG. 2 illustrates schematically another embodiment of the electrical stimulation system 100, where the lead 103 is a percutaneous lead. In FIG. 2, the electrodes 134 are shown disposed along the one or more lead bodies 106. In at least some embodiments, the lead 103 is isodiametric along a longitudinal length of the lead body 106.

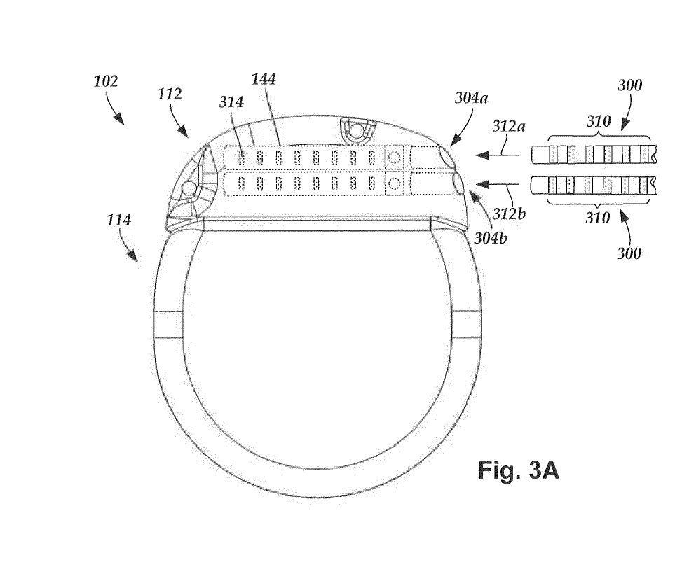

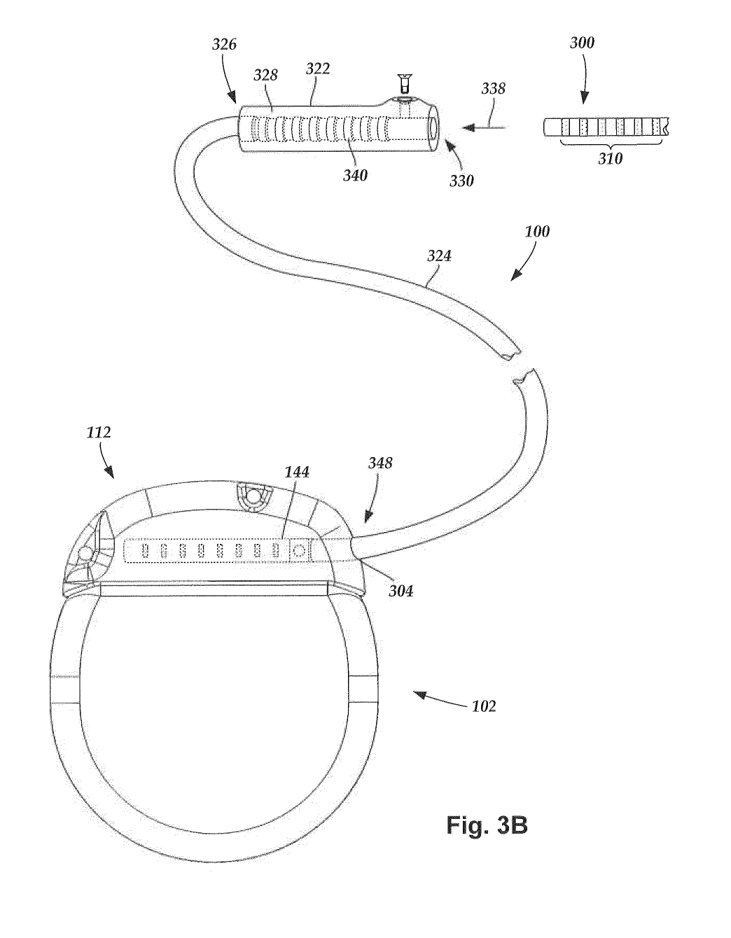

The lead 103 can be coupled to the control module 102 in any suitable manner. In FIG. 1, the lead 103 is shown coupling directly to the control module 102. In at least some other embodiments, the lead 103 couples to the control module 102 via one or more intermediate devices (300 in FIGS. 3A-3B). For example, in at least some embodiments one or more lead extensions 324 (see e.g., FIG. 3B) can be disposed between the lead 103 and the control module 102 to extend the distance between the lead 103 and the control module 102. Other intermediate devices may be used in addition to, or in lieu of, one or more lead extensions including, tor example, a splitter, an adaptor, or the like or combinations thereof. It will be understood that, in the case where the electrical stimulation system 100 includes multiple elongated devices disposed between the lead 103 and the control module 102, the intermediate devices may be configured into any suitable arrangement.

In FIG. 2, the electrical stimulation system 100 is shown having a splitter 207 configured and arranged for facilitating coupling of the lead 103 to the control module 102. The splitter 207 includes a splitter connector 208 configured to couple to a proximal end of the lead 103, and one or more splitter tails 209a and 209b configured and arranged to couple to the control module 102 (or another splitter, a lead extension, an adaptor, or the like).

The control module 102 typically includes a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

The electrical stimulation system or components of the electrical stimulation system, including the paddle body 104, the one or more of the lead bodies 106, and the control module 102, are typically implanted into the body of a patient. The electrical stimulation system can be used for a variety of applications including, but not limited to deep brain stimulation, neural stimulation, spinal cord stimulation, muscle stimulation, and the like.

The electrodes 134 can be formed using any conductive, biocompatible material. Examples of suitable materials include metals, alloys, conductive polymers, conductive carbon, and the like, as well as combinations thereof in at least some embodiments, one or more of the electrodes 134 are formed from one or more of: platinum, platinum iridium, palladium, palladium rhodium, or titanium.

Any suitable number of electrodes 134 can be disposed on the lead including, for example, four, five, six, seven, eight, nine, ten, eleven, twelve, fourteen, sixteen, twenty-four, thirty-two, or more electrodes 134. In the case of paddle leads, the electrodes 134 can be disposed on the paddle body 104 in any suitable arrangement. In FIG. 1, the electrodes 134 are arranged into two columns, where each column has eight electrodes 134.

The electrodes of the paddle body 104 (or one or more lead bodies 106) are typically disposed in, or separated by, a non-conductive, biocompatible material such as, tor example, silicone, polyurethane, polyetheretherketone (“PEEK”), epoxy, and the like or combinations thereof. The one or more lead bodies 106 and, if applicable, the paddle body 104 may be formed in the desired shape by any process including, for example, molding (including injection molding), casting, and the like. The non-conductive material typically extends from the distal ends of the one or more lead bodies 106 to the proximal end of each of the one or more lead bodies 106.

In the case of paddle leads, the non-conductive material typically extends from the paddle body 104 to the proximal end of each of the one or more lead bodies 106. Additionally, the non-conductive, biocompatible material of the paddle body 104 and the one or more lead bodies 106 may be the same or different. Moreover, the paddle body 104 and the one or more lead bodies 106 may be a unitary structure or can be formed as two separate structures that are permanently or detachable coupled together.

Terminals (e.g., 310 in FIGS. 3A-3B) are typically disposed along the proximal end of the one or more lead bodies 106 of the electrical stimulation system 100 (as well as any splitters, lead extensions, adaptors, or the like) for electrical connection to corresponding connector contacts (e.g., 314 in FIGS. 3A-3B). The connector contacts are disposed in connectors (e.g., 144 in FIGS. 1-3B; and 322 FIG. 3B) which, in turn, are disposed on, for example, the control module 102 (or a lead extension, a splitter, an adaptor, or the like). Electrically conductive wires, cables, or the like (not shown) extend from the terminals to the electrodes 134. Typically, one or more electrodes 134 are electrically coupled to each terminal. In at least some embodiments, each terminal is only connected to one electrode 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably scalable at the distal end.

FIG. 3A is a schematic side view of one embodiment of a proximal end of one or more elongated devices 300 configured and arranged for coupling to one embodiment of the control module connector 144. The one or more elongated devices may include, for example, one or more of the lead bodies 106 of FIG. 1, one or more intermediate devices (e.g., a splitter, the lead extension 324 of FIG. 3B, an adaptor, or the like or combinations thereof), or a combination thereof.

The control module connector 144 defines at least one port into which a proximal end of the elongated device 300 can be inserted, as shown by directional arrows 312a and 312b. In FIG. 3A (and in other figures), the connector housing 112 is shown having two ports 304a and 304b. The connector housing 112 can define any suitable number of ports including, for example, one, two, three, four, five, six, seven, eight, or more ports.

The control module connector 144 also includes a plurality of connector contacts, such as connector contact 314, disposed within each port 304a and 304b. When the elongated device 300 is inserted into the ports 304a and 304b, the connector contacts 314 can be aligned with a plurality of terminals 310 disposed along the proximal end(s) of the elongated device(s) 300 to electrically couple the control module 102 to the electrodes (134 of FIG. 1) disposed on the paddle body 104 of the lead 103, Examples of connectors in control modules are found in, for example, U.S. Pat. Nos. 7,244,150 and 8,224,450, which are incorporated by reference.

FIG. 3B is a schematic side view of another embodiment of the electrical stimulation system 100. The electrical stimulation system 100 includes a lead extension 324 that is configured and arranged to couple one or more elongated devices 300 (e.g., one of the lead bodies 106 of FIGS. 1 and 2, the splitter 207 of FIG. 2, an adaptor, another lead extension, or the like or combinations thereof) to the control module 102. In FIG. 3B. the lead, extension 324 is shown coupled to a single port 304 defined in the control module connector 144. Additionally, the lead extension 324 is shown configured and arranged to couple to a single elongated device 300. In alternate embodiments, the lead extension 324 is configured and arranged, to couple to multiple ports 304 defined in the control module connector 144, or to receive multiple elongated devices 300, or both.

A lead extension connector 322 is disposed on the lead extension 324. In FIG. 3B, the lead extension connector 322 is shown disposed at a distal end 326 of the lead extension 324. The lead extension connector 322 includes a connector housing 328. The connector housing 328 defines at least one port 330 into which terminals 310 of the elongated device 300 can be inserted, as shown by directional arrow 338. The connector housing 328 also includes a plurality of connector contacts, such as connector contact 340, When the elongated device 300 is inserted into the port 330, the connector contacts 240 disposed in the connector housing 328 can be aligned with the terminals 310 of the elongated device 300 to electrically couple the lead extension 324 to the electrodes (134 of FIGS. 1 and 2) disposed along the lead (103 in FIGS. 1 and 2).

In at least some embodiments, the proximal end of the lead extension 324 is similarly configured and arranged as a proximal end of the lead 103 for other elongated device 300). The lead extension 324 may include a plurality of electrically conductive wires (not shown) that electrically couple the connector contacts 340 to a proximal end 348 of the lead extension 324 that is opposite to the distal end 326. In at least some embodiments, the conductive wires disposed in the lead extension 324 can be electrically coupled to a plurality of terminals (not shown) disposed along the proximal end 348 of the lead extension 324. In at least some embodiments, the proximal end 348 of the lead extension 324 is configured and arranged for insertion into a connector disposed in another lead extension (or another intermediate device), in other embodiments (and as shown in FIG. 3B), the proximal end 348 of the lead extension 324 is configured and arranged for insertion into the control module connector 144.

A lead can be anchored in patient tissue using a lead anchor. More particularly, a lead anchor can be designed so that the lead is side loaded into the lead anchor in contrast to many conventional lead anchors that are slid onto the lead starting at either the proximal or distal end of the lead. This is particular advantageous for leads that are not isodiametric or which are bifurcated or branched. In such lead, it may be difficult to slide a lead anchor along the lead.

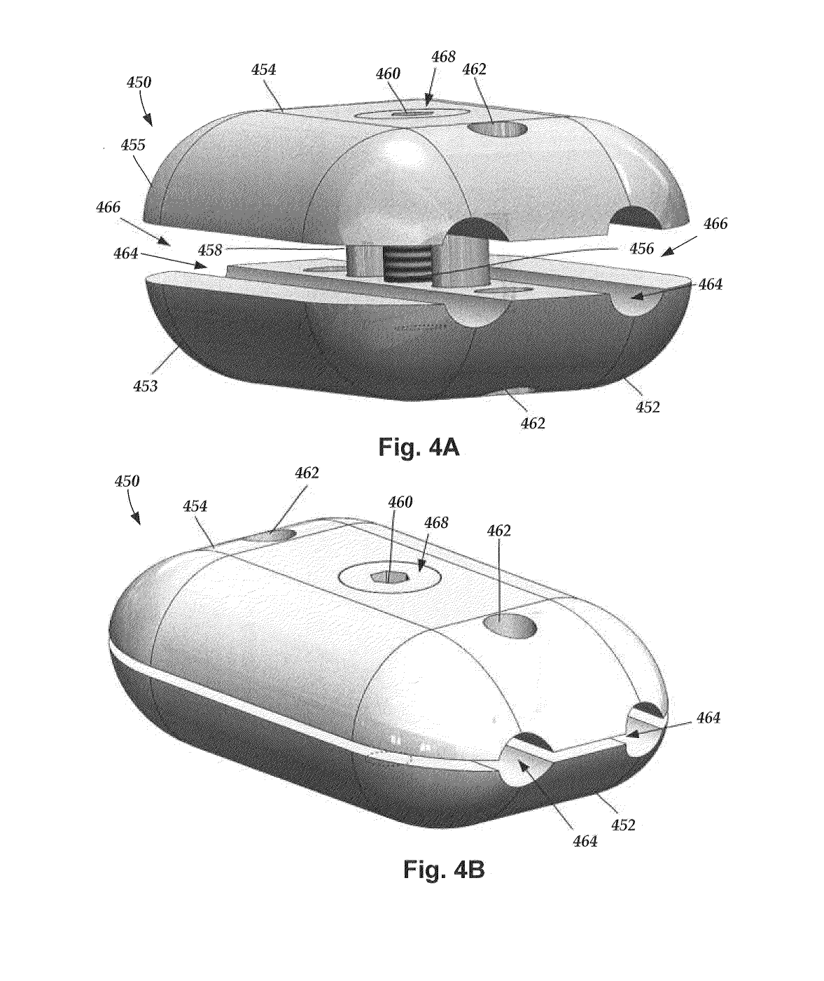

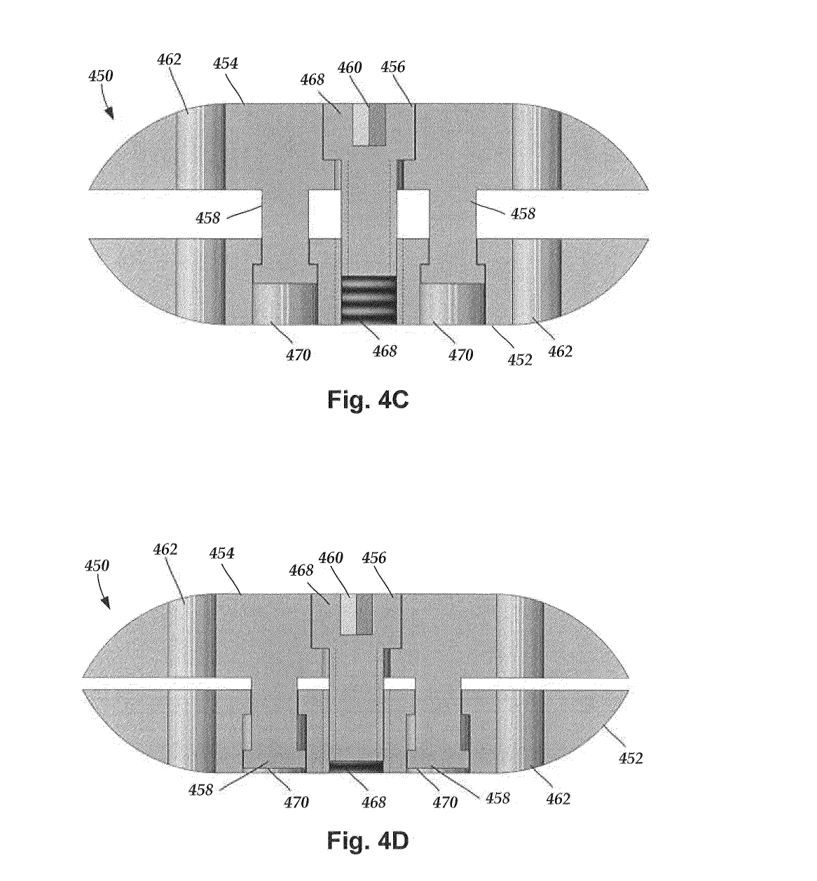

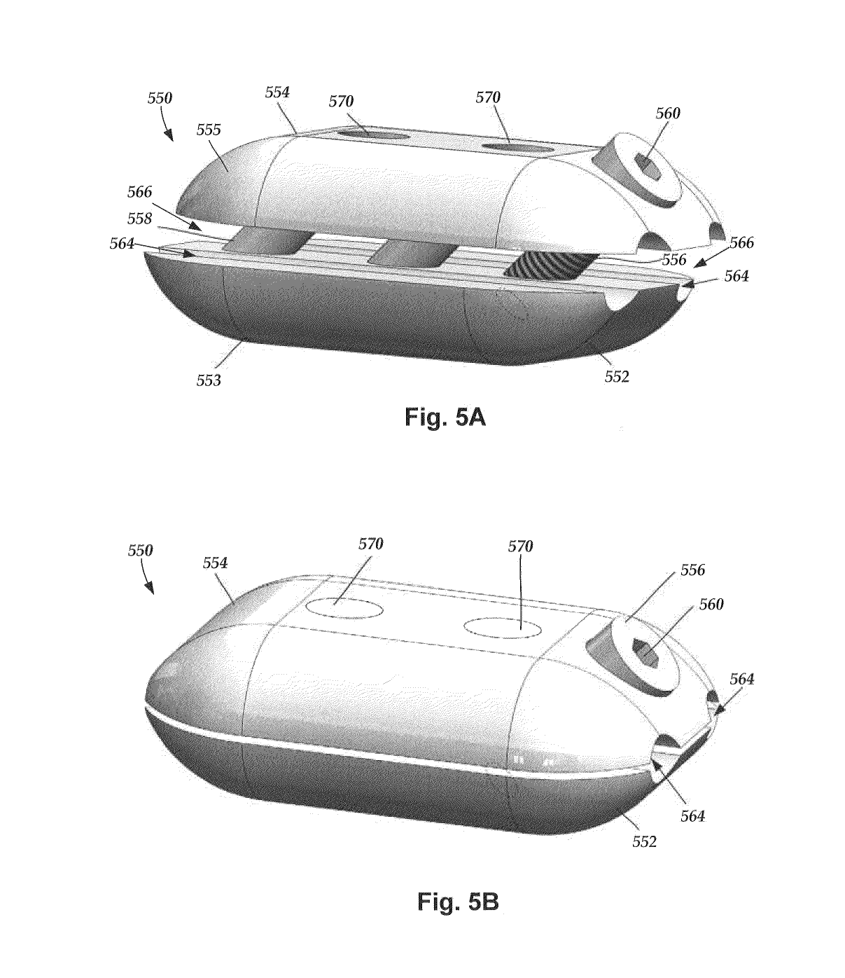

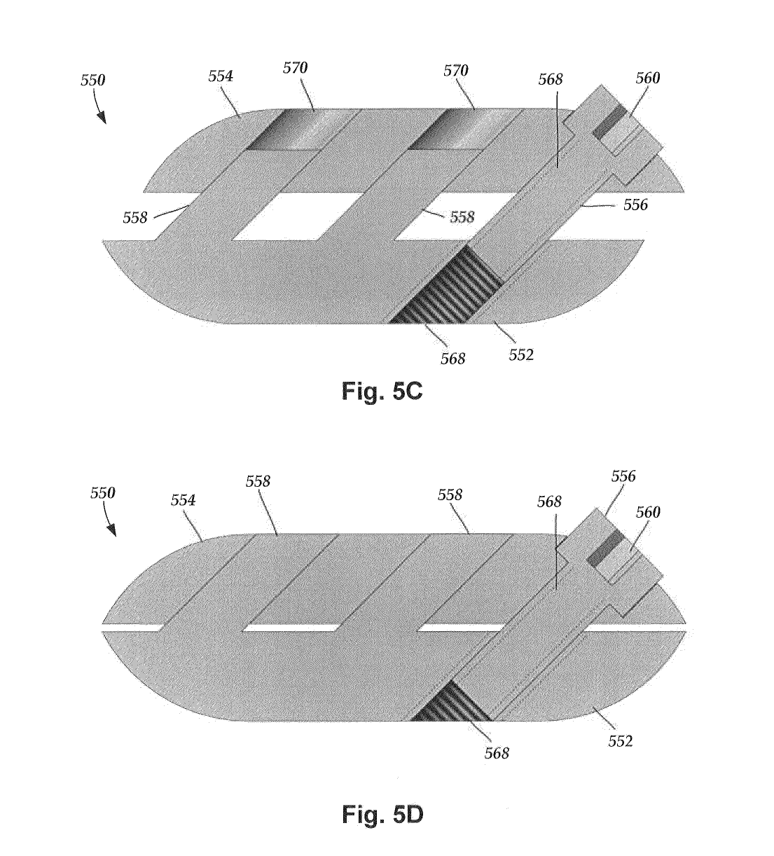

After loading the lead, lead movement is constrained when the lead anchor is closed via the turning of a screw or other fastener. FIGS. 4A-4D illustrate one embodiment of the lead anchor and FIGS. 5A-5D illustrate another embodiment of the lead anchor.

FIG. 4A illustrates one embodiment of a dual side-loading lead anchor in a load position. The implantable lead anchor 450 includes two anchor shells 452, 454 disposed opposite each other. Each of the anchor shells 452, 454 is made of any suitable biocompatible material, for example, a plastic or polymer, such as, silicone, poly vinylchloride, polyurethane, or the like; a biocompatible metal or alloy, such as titanium or titanium alloys, nickel, aluminum, stainless steel, copper, gold, silver, platinum or alloys thereof; or any other suitable biocompatible material or combination of materials. In some embodiments, the anchor shells 452, 454 are made of the same material, such as silicone. In some embodiments, the first anchor shell 452 and the second anchor shell 454 are made of different materials. Furthermore, it may be useful for any or all parts of the lead anchor 450 to be made of or incorporate, a radiopaque material, so that it is visible using fluoroscopy or other forms of X-ray imaging.

In some embodiments, the anchor shells 452, 454 may have a shape that is substantially a cube or parallel-piped. It will be recognized, however, that other shapes are also suitable including a cylindrical shape or a shape like a gastropod shell that is oblong, conical, depressed, or the like. Each of the sides 453 of the anchor shell can be rounded, The rounded shape of the sides 453 can reduce damage to body tissue, thereby ensuring safety during both installation and use. In some embodiments, the anchor shells 452, 454 have the same shape. In other embodiments, the anchor shells can have different shapes or different cross-sections.

In at least some embodiments, each of the anchor shells 452, 452 has a width greater than its height. Here, height of the anchor shell is measured as a maximum measurement along a central axis passing along a vertical length of the shell when oriented as illustrated in FIG. 4A. Width is measured as a maximum measurement along a horizontal length of the shell when oriented as illustrated in FIG. 4A. In some embodiments, each of the anchor shells 452, 454 has corresponding width less than, or equal to that of the corresponding height.

Each of the anchor shells 452, 454 has at least two lead channels 464 formed on a surface of the anchor shell. In at least some embodiments, the lead channels are parallel to each other. In at least some embodiments, the lead channels 464 can be defined by as a depression in the surface of the anchor shell. In at least some embodiments, the lead channel 464 is a groove extending along a longitudinal length of the anchor shell. The lead channel 464 may be substantially dimensioned to receive the lead laterally and form a friction fit with the lead. Typically, corresponding lead channels 464 on the first anchor shell 452 and second anchor shell 454 align with each other.

Each lead channel 464 may have a cross-section that is partially circular as it extends along the anchor shell. It is contemplated that the lead channel 464 may also have a cross-section in the shape of a triangle, a square, an ovoid, or substantially like a groove, or furrow, or any other suitable shape that is large enough to house the lead.

In the illustrated embodiment, two lead channels 464 are employed to grip/house two leads such that one lead of the two leads passes through one lead channel 464 of the two lead channels 464 and another lead of the two leads passes through another lead channel 464 of the two lead channels 464. In some embodiments, each of the two anchor shells 452, 454 contains more than two lead channels so that the lead anchor 450 is able to grip/house more than two leads at a time. In some embodiments, the lead and the lead anchor 450 form a kit.

The lead channel 464 can anchor isodiametric leads. In some embodiments, the lead channel 464 also allows for gripping/housing of the leads which are non-isodiametric, bifurcated, branched, or the like. For example, the lead anchor 450 houses two non-isodiametric leads by disposing portions of each of the non-isodiametric leads into each of the two parallel lead channels 464.

In some embodiments, the lead channel 464 is defined so that the lead passes along a straight path formed in the anchor shells 452, 454. In other embodiments, the lead channel 464 may be defined so that the lead passes at an angled path formed in the anchor shells 452, 454. In some embodiments, the lead channel 464 may be defined as a curved or non-linear path formed in the anchor shells 452, 454. In some embodiments, the lead channel 464 is formed by molding, piercing, boring, reaming, tapping, or any other suitable manufacturing method, in some embodiments, the lead channel 464 may include interior threads, ridges, micro patterns, or another suitable roughening of the surface of the channel for better engagement with the lead.

The two anchor shells 452, 454 are spaced apart to form a side loading aperture 466, The side loading aperture 466, when the lead anchor is in the load position, as illustrated in FIG. 4A, permits the lead anchor to laterally receive a lead that can then be positioned within the opposing lead channels 464 of the anchor shells 452, 454. When the lead anchor 450 is closed to a lock position, the lead channels 464 (disposed opposite each other) come closer to each other and the side loading aperture 466 becomes narrower so that the lead cannot be removed from the lead anchor.

The first anchor shell 452 and the second anchor shell 454 further include an aperture 468 to receive an actuating mechanism such as a screw 456. In some embodiments, other actuating mechanisms such as a pin, nail, dowel, rod, or any combination thereof or any other suitable item for engaging and anchoring the anchor shells 452, 454 can be used. The aperture 468 is typically cylindrical, but it will be understood that apertures with other shapes can be used including, but not limited to those with cross-sections in the shape of a triangle, rectangle, a square, an ovoid, or any other suitable shape that is capable of receiving a movable actuating mechanism. In at least some embodiments, the aperture is positioned between the two parallel lead channels 464 of the anchor shells 452, 454.

The aperture 468 can extend entirely through the anchor shell 452, 454, or, in the case of first anchor shell 452, the aperture may not extend all of the way to the exterior surface of the shell 452. In some embodiments, the aperture 468 may be formed by molding, boring, reaming, or tapping the anchor shells 452, 454. The screw 456 or other actuating mechanism extends from the second anchor shell 454 to the first anchor shell 452, or vice versa, thereby holding the first anchor shell 452 and the second anchor shell 454 together.

The apertures 468 may have a thread, groove, crease, channel, duct, or rib, or the like, fur facilitating movement of the screw 456 and maintaining the position of the screw 456 within the aperture. In some embodiments, there is a plurality of apertures to receive more than one screw 456 or other actuating mechanism.

The screw 456 can include two parts—a head and a tail. Thus, the screw 456 may substantially exhibit a T-shape in cross-section. The head portion of the screw 456 can have a cross-section that is substantially circular. However, the head portion of the screw 456 may have other cross-sectional shapes, such as a rectangular, an oblong, an oval, an elliptical, a triangular, or a hexagonal cross-section, or the like. A top surface of the head portion of the screw 456 can have a single slit or two slits intersecting each other orthogonally. In some embodiments, the bead portion has a plurality of slits or other aperture that can receive a tool and has a cross-section in the shape of for example, a rectangle, a star, a square, a polygon, or the like. Thus, the top surface of the head portion forms a screw head aperture 460 to receive a tip of a tool such that the tool can be turned in a clockwise direction and a counter-clockwise direction (or vice versa) to tighten and loosen the screw 456, respectively, in some embodiments, the tool is a torque-limiting tool to prevent or reduce damage to the lead anchor as the screw or other actuating mechanism is operated. The actuating mechanism, such as screw 456, can be operated to move the anchor shells 452, 454 together or apart, by any method including, but not limited to, tightening, screwing, pushing/pulling, or the like. In some embodiments, the tool is a screwdriver, wrench, pliers, or drill or any other suitable tool useful for setting, fixing, screwing, tightening, fastening or fitting the actuating mechanism with the lead anchor 450.

In at least some embodiments, a substantial portion of the tail portion of the screw 456 is threaded such that threaded tail portion of the screw 456 is received in a threaded aperture 46% of the anchor shells 452, 454. A portion of the aperture 468 in the second anchor shell 454 may have dimensions different than the remainder of the aperture to receive the head portion of the screw 456. The remainder of the aperture, as well as the aperture 468 in the first anchor shell 452, is dimensioned to receive the tail portion of the screw 456. However, in some embodiments, both such apertures can be of same diameter and in such case the screw 456 can have head and tail portions of same diameter. Thus, diameters of both the apertures depend upon the diameter of the actuating mechanism used.

In at least some embodiments, one or both of the first anchor shell 452 and the second anchor shell 454 further define one or more suture openings 462 through, the two anchor shelf The suture openings 462 allow a suture (not shown) to extend through the suture openings to suture the lead anchor to patient tissue. A suture inserter such as a needle, or the like can be used to introduce and advance the suture into the suture opening 462, The diameter of the suture opening 462 may depend on thickness of the suture or the suture inserter. In some embodiments, the suture is a thread, wire, silk, or any other suitable member that has capability to suture the lead anchor 450 of the tissue.

The lead anchor 450 further includes at least one guide element 458 that extends between the first anchor shell 452 and the second anchor shell 454. The guide elements 458 are employed to maintain an orientation between the two anchor shells 452, 454. The guide elements 458 may have a cylindrical shape. It is contemplated that the guide element 458 may also have a cross-section in the shape of a circle, a triangle, a square, an ovoid, or any other suitable shape that is able to maintain an orientation between the two anchor shells 452, 454.

The guide element 458 may be made of a similar material as that of the anchor shells 452, 454 or a different material. Example of suitable materials for the guide element 458 include, but are not limited to, a metal or alloy, such as titanium or titanium alloys, nickel, platinum, cobalt, gold, silver and alloys thereof or any other biocompatible metal, or a rigid plastic, silicone, or polymer, or the like.

The first anchor shell 452 and the second anchor shell 454 as shown in FIG. 4A are in a load position. In the load position, the first anchor shell 452 and the second anchor shell 454 are spaced apart such that a portion of the lead can be laterally inserted through the side loading aperture 466 of the lead channel 464. In the illustrated embodiment, the lead anchor can receive two leads laterally so the illustrated lead anchor is dual side-loading.

FIG. 4B illustrates the dual side-loading lead anchor 450 of FIG. 4A in a lock position. In the lock position, the first anchor shell 452 and the second anchor shell 454 are brought closer together than in the load position using the actuating mechanism, such as screw 456. This locks the lead(s) in the lead anchor 450.

FIG. 4C is a cross-sectional view of the dual side-loading lead anchor of FIG. 4A in the load position. As shown in FIG. 4C, the first anchor shell 452 includes an aperture 468 configured to receive a tail of the screw 456. The aperture 468 defines a transverse lumen in which the screw 456 is tightened. The transverse lumen may have a cross-section that is substantially circular, in other embodiments, the aperture 468 defines a transverse lumen with a cross-section in the shape of a triangle, a square, an ovoid, or any other suitable shape that is capable of housing the screw 456. Furthermore, the transverse lumen may have a thread, groove, crease, channel, duct, or rib, or the like, for facilitating and accepting the tail portion of the screw 456, in some embodiments, the screw 456 is aligned perpendicularly with respect to the two parallel lead channels and the transverse lumen of the aperture 468 is positioned perpendicular to the central axis of the lead, anchor 450.

On both sides of the aperture 468, two apertures 470 in first anchor shell 452 are disposed to receive the guide elements 458. Each of the apertures 470 is receives a respective guide element 458. As shown in FIG. 4C, the guide elements 458 protrude from second anchor shell 454 and reside in apertures defined in first anchor shell 452. It will be understood that the opposite arrangement is also available as well as an arrangement in which one guide element protrudes from the first anchor shell 452 and the other guide element protrudes from the second anchor shell with corresponding apertures in the opposite anchor shell.

Further, in at least some embodiments, as illustrated in FIGS. 4C and 4D, the guide elements 458 include heads with a larger diameter than the adjacent shafts and each of the apertures 470 include a stop that prevents extraction of the guide elements from the apertures by blocking the head of the guide element. This arrangement prevents the two anchor shells 452, 454 from totally disengaging from each other.

The aperture 470 may be open at the exterior surface of the first anchor shell 452 or may be closed, The aperture 470 may have a cross-section that is substantially circular. However the aperture 470 may define a transverse lumen with a cross-sectional shape that is rectangular, ovoid, square, or any combinations thereof, capable of receiving the guide element 458.

Since there are two apertures 470 in the first anchor shell 452, and tints two guide elements, a first guide element and a second guide element, are received in respective ones of the two apertures 470. It will be understood that there can be more than two guide elements 458 and more than two apertures 470 to receive respective guide elements 458.

FIG. 4D is a cross-sectional view of the dual side-loading lead anchor 450 of FIG. 4A in the lock position. As depicted in FIG. 4D, the two anchor shells 452, 454 are brought closer together such that the anchor shells clamp down on the lead(s) to lock it in place. The screw 456 is tightened by turning the tool, in at least some embodiments, the tool is a torque limiting tool set to a certain threshold above which it will no longer tighten the screw 456. For example, the tool is configured to Limit the number of revolutions or the depth to which the screw 456 is advanced. Consequently, over tightening of the screw 456 is avoided, and the lead can be protected from possible damage due to over tightening of the screw 456.

In at least some embodiments, the anchor can consist of three components: the screw 456, die first anchor shell 452, and the second anchor shell 454. Thus, each of first anchor shell 452 and the second anchor shell 454 can be formed (machined, molded, etc.) from a single and continuous piece of material. Alternatively, each of the components of the first anchor shell 452 and the second anchor shell 454 can be made from separate pieces of the same or different material and attached to the first anchor shell 452 or the second anchor shell 454. For example, the guide elements 458 could be separately attached to one of the first anchor shell 452 or the second anchor shell 454 by welding, screwing, adhering, or other attachment methods. In addition, although a suture opening 462 is contemplated, other structures or materials can be provided tor attaching the lead anchor 450 to a patient. For example, a netting or looping about a periphery of the lead anchor 450 can be provided. For suturing the lead anchor 450 to a patient or there can be grooves in the exterior surfaces of the anchor shells for receiving sutures wrapped around the anchor shells. In addition, an adhesive surface or barb surface can be provided on the lead anchor 450 to allow it to be attached to a patient. A material can also be provided that allows the lead anchor 450 to be stapled to tissue.

FIG. 5A illustrates another embodiment of a dual side-loading lead anchor 550 in a load position. The lead anchor 550 includes a first anchor shell 552 and a second anchor shell 554 disposed opposite the first anchor shell 552. Each of the two anchor shells 552, 554 defines two parallel lead channels 564 and side loading apertures 566. Further, the first anchor shell 552 has its sides 553 of arcuate shape. Likewise, the second anchor shell 554 has us sides 555 of arcuate shape. Except as described below, the design and other considerations described for lead anchor 450 also apply to lead anchor 550.

Although the structure of the lead anchor 550 is similar to that of the lead anchor 450 of FIG. 4A, the actuating mechanism (e.g., screw 556) and corresponding aperture 558 is aligned at art acute angle with respect to the two parallel lead channels 564. In at least some embodiments, the acute angle is in a range from 20 to 70 degrees. The head portion of the screw 556 includes a screw bead aperture 560 to receive a tip of a tool. Furthermore, in this embodiment, the guide elements 558, and their associate apertures 570, may also be aligned at the acute angle with respect to the two parallel lead channels 564. Furthermore, the leach anchor may include suture opening(s) (not shown) to suture the lead anchor 550 to the tissue.

FIG. 5B illustrates the dual side-loading 550 of FIG. 5A in a lock position. In the lock position, the first anchor shell 552 and the second anchor shell 554 are brought closer together such that lead channels 564 of both the anchor shells 552, 554 clamp down on the lead to hold it within the lead anchor.

FIG. 5C is a cross-sectional view of the dual side-loading lead anchor 550 of FIG. 5A in the load position. This figures also illustrates that the screw head aperture 560 is aligned at the acute angle with respect to the lead channels 564. The second anchor shell 554 includes two apertures 570 to receive the guide elements 558 with the apertures and guide elements also aligned at the acute angle.

FIG. 5D is a cross-sectional view of the dual side-loading lead anchor 550 of FIG. 5A in the lock position. It should be noted that during the locking operation of the first anchor shell 552 with respect to the second anchor shell 554, the first anchor shell 552 will move in an angular two dimensional direction (both towards and along) with respect to the second anchor shell 554. This embodiment is different from the embodiment of FIG. 4A, where the relative movement will be substantially orthogonal and one dimensional (towards) with respect to the second anchor shell 554.

A method of implanting the electrical stimulation lead includes a number of consecutive, non-consecutive, simultaneous, non-simultaneous, or alternative steps. For instance, the method includes provide the lead anchor with the first and second anchor shells in the load, position. A portion of one or more stimulation leads is side-loaded, through the side loading apertures 466 and into one of the lead channels. Thereafter, the screw 456 is tightened to adjust the lead anchor from the load position to the lock position, in the lock position, both the anchor shells clamp down on the lead to hold it in place. Further, the lead anchor can be sutured to tissue by passing a suture through the suture opening(s), it will be recognized that the lead anchor may also be operated to release the lead(s) by moving from the lock position to the load position.

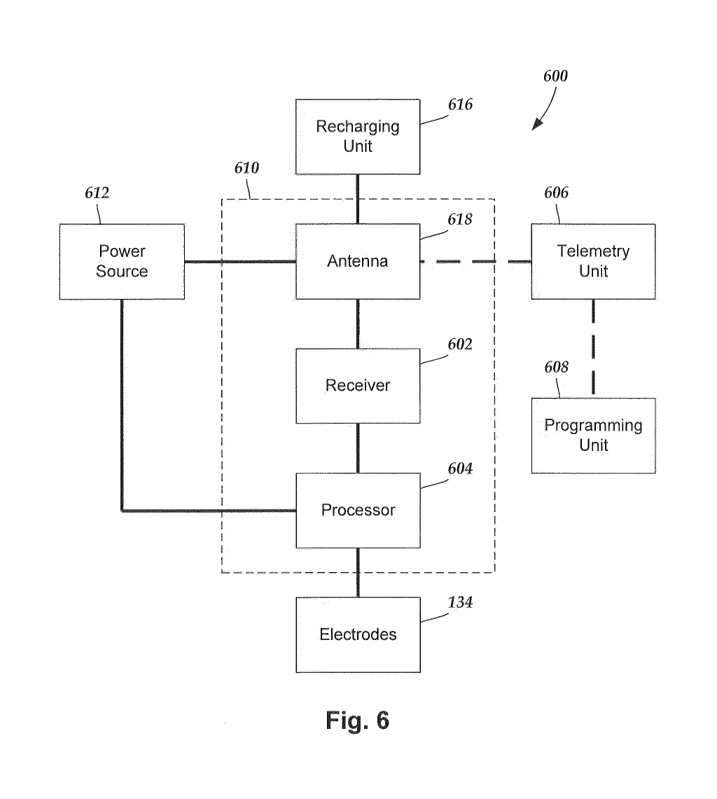

FIG. 6 is a schematic overview of one embodiment of components of an electrical stimulation system 600 including an electronic subassembly 610 disposed within a control module. It will be understood that the electrical stimulation system can include more, fewer, or different components and can have a variety of different configurations including those configurations disclosed in the stimulator references cited herein.

Some of the components (for example, a power source 612, an antenna 618, a receiver 602, and a processor 604) of the electrical stimulation system can be positioned on one or more circuit boards or similar carriers within a sealed housing of an implantable pulse generator, if desired. Any power source 612 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 618 or a secondary antenna. The external power source can be in a device that is mounted on the skin of the user or in a unit that is provided near the user on a permanent or periodic basis.

If the power source 612 is a rechargeable battery, the battery may be recharged using the optional antenna 618, if desired. Power can be provided to the battery for recharging by inductively coupling the battery through the antenna to a recharging unit 616 external to the user. Examples of such arrangements can be found in the references identified above.

In one embodiment, electrical current is emitted by the electrodes 134 on the paddle or lead body to stimulate nerve fibers, muscle fibers, or other body tissues near the electrical stimulation system. The processor 604 is generally included to control the timing and electrical characteristics of the electrical stimulation system. For example, the processor 604 can, if desired, control one or more of the timing, frequency, strength, duration, and waveform of the pulses. In addition, the processor 604 can select which electrodes can be used to provide stimulation, if desired. In some embodiments, the processor 604 selects which electrode(s) are cathodes and which electrode(s) are anodes. In some embodiments, the processor 604 is used to identify which electrodes provide the most useful stimulation of the desired tissue.

Any processor can be used and can be as simple as an electronic device that, for example, produces pulses at a regular interval or the processor can be capable of receiving and interpreting instructions from an external programming unit 608 that, for example, allows modification of pulse characteristics. In the illustrated embodiment, the processor 604 is coupled to a receiver 602 which, in turn, is coupled to the optional antenna 618. This allows the processor 604 to receive instructions from an external source to, for example, direct the pulse characteristics and the selection of electrodes, if desired.

In one embodiment, the antenna 618 is capable of receiving signals (e.g., RF signals) from an external telemetry unit 606 which is programmed by the programming unit 608, The programming unit 608 can be external to, or part of the telemetry unit 606. The telemetry unit 606 can be a device that is worn on die skin of the user or can be carried by the user and can have a form similar to a pager, cellular phone, or remote control, if desired. As another alternative, the telemetry unit 606 may not be worn or carried by the user but may only be available at a home station or at a clinician's office. The programming unit 608 can be any unit that can provide information to the telemetry unit 606 for transmission (o the electrical stimulation system 600. The programming unit 608 can be part of the telemetry unit 606 or can provide signals or information to the telemetry unit 606 via a wireless or wired connection. One example of a suitable programming unit is a computer operated by the user or clinician to send signals to the telemetry unit 606.

The signals sent to the processor 604 via the antenna 618 and the receiver 602 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct die electrical stimulation system 600 to cease operation, to start operation, to start charging the battery, or to stop charging the battery. In other embodiments, the stimulation system does not include the antenna 618 or receiver 602 and the processor 604 operates as programmed.

Optionally, the electrical stimulation system 600 may include a transmitter (not shown) coupled to the processor 604 and the antenna 618 for transmitting signals back to the telemetry unit 606 or another unit capable of receiving the signals. For example, the electrical stimulation system 600 may transmit signals indicating whether the electrical stimulation system 600 is operating properly or not or indicating when the battery needs to be charged or the level of charge remaining in the battery. The processor 604 may also be capable of transmitting information about the pulse characteristics so that a user or clinician can determine or verify the characteristics.

The above specification, examples and data provide a description of the manufacture and use of the composition of the invention. Since many embodiments of the invention can be made without departing from the spirit and scope of the invention, the invention also resides in the claims hereinafter appended.

What is claimed as new and desired to be protected by Letters Patent of the United States is:

1. An implantable lead anchor, comprising:
a first anchor shell;
a second anchor shell disposed opposite the first anchor shell, the first and second anchor shells defining two parallel lead channels between the first and second anchor shells;
an actuating mechanism to operable adjust the first and second anchor shells between a load position and a lock position, wherein, in the lock position, the first and second anchor shells are closer together than in the load position and any lead disposed within one of the two parallel lead channels is locked in place and, in the load position, the first and second anchor shells are sufficiently spaced apart so that a portion of a lead can be side-loaded between the first and second anchor shells and into one of the two parallel lead channels; and
at least one guide element that extends between the first and second anchor shells and is configured and arranged to maintain an orientation of the first and second anchor shells relative to each other in the lock position, in the load position, and during adjustment between the lock and load positions.

2. The implantable lead anchor of claim 1, wherein the actuating mechanism comprises a screw extending from the first anchor shell to the second anchor shell.

3. The implantable lead anchor of claim 2, wherein the first anchor shell comprises a threaded hole configured and arranged to receive a threaded portion of the screw.

4. The implantable lead anchor of claim 2, wherein the screw is perpendicularly aligned with respect to the two parallel lead channels.

5. The implantable lead anchor of claim 2, wherein the screw is aligned at an acute angle with respect to the two parallel lead channels.

6. The implantable lead anchor of claim 2, wherein the acute angle is in a range from 20 to 70 degrees.

7. The implantable lead anchor of claim L wherein the at least one guide element is two guide elements.

8. The implantable lead anchor of claim 7, wherein the actuating mechanism comprises a screw extending from the first anchor shell to the second anchor shell and the screw is disposed between the two guide elements.

9. The implantable lead anchor of claim 7, wherein the actuating mechanism comprises a screw extending from the first anchor shell to the second anchor shell and the two guide elements comprise a first guide element and a second guide elements, wherein the second guide element is disposed between the first guide element and the screw.

10. The implantable lead anchor of claim 7, wherein the two guide elements are posts extending from the first anchor shell and the second anchor shell defines two apertures that receive the posts.

11. The implantable lead anchor of claim 1, wherein each of the first and second shells defines at least one suture opening configured and arranged to allow a suture to extend through suture openings of the first and second shells for suturing the implantable lead anchor to tissue.

12. The implantable lead anchor of claim 1, wherein the actuating mechanism and the at least one guide element extend through portions of the first and second shells disposed between the two parallel lead channels defined in the first and second shells.

13. A kit, comprising:
the implantable lead anchor of claim 1; and
at least one electrical stimulation lead, wherein the implantable lead anchor is configured and arranged to receive a portion of one of the at least one electrical stimulation lead in one of the two parallel lead channels.

14. The kit of claim 13, further comprising a control module coupleable to the at least one electrical stimulation lead.

15. The kit of claim 13, wherein that at least one electrical stimulation lead is two electrical stimulation leads.

16. A method of implanting an electrical stimulation lead, the method comprising:
side-loading a portion of a first electrical, stimulation lead into one of the two lead channels of the implantable lead anchor of claim 1, wherein the first and second anchor shells of the implantable lead anchor are in the load position; and
operating the actuating mechanism to adjust the first and second anchor shells to the lock position to lock the first electrical stimulation lead in the implantable lead anchor.

17. The method of claim 16, further comprising side-loading a portion of a second electrical stimulation lead into another one of the two lead channels of the implantable lead, anchor when the first and second shells are in the load position.

18. The method of claim 16, wherein the actuating mechanism comprises a screw extending from the first anchor shell to the second anchor shell and wherein operating the actuating mechanism comprises using a tool to rotate the screw causing the first and second anchor shells to adjust from the load position to the lock position.

19. The method of claim 18, wherein the screw is aligned at an acute angle with respect to the two parallel lead channels.

20. The method of claim 16, wherein each of the first and second shells defines at least one suture opening, the method further comprising suturing the implantable lead anchor to tissue using the at least one suture opening of each of the first and second shells.
